(19) United States
(12) Patent Application Publication
(10) Pub. No.: US 2021/0267546 A1
(43) Pub. Date: Sep. 2, 2021

(54) EAR TEMPERATURE MEASUREMENT OF WEARABLE HEADSET

(71) Applicant: Kai-Chun CHEN, New Taipei City (TW)

(72) Inventor: Kai-Chun CHEN, New Taipei City (TW)

(21) Appl. No.: 17/024,741

(22) Filed: Sep. 18, 2020

(30) Foreign Application Priority Data
Mar. 2, 2020 (TW) 109202278

(51) Int. Cl. A61B 5/00; A61B 5/01

(52) U.S. Cl. CPC A61B 5/6803; A61B 5/0008; A61B 5/01; A61B 5/7475; A61B 5/002; A61B 5/7435; A61B 5/0086

(57) ABSTRACT

Besides of a forehead thermometer and a thermography imaging equipment, other temperature measuring tools requires a direct contact with the human bodies, however, the forehead thermometer used for measuring is in a close distance, this time consuming and an additional risk of getting infected will happen; the thermography imaging equipment has an advantage of rapidly measuring, but the cost thereof is high and could not be prevailed; as such, the present invention is to consider a product combining a temperature measuring tool and a wearable headset, and a wired or a wireless means is used for transmitting data with a smart phone, a virtual button of an APP or a button module of the headset can switch functions, the relevant data is displayed on a screen of the smart phone, moreover, the measured temperature is uploaded to a cloud for allowing a quarantine inspector to perform an initial termination.

### BACKGROUND OF THE INVENTION

### 1. Field of the Invention

Droplet contact is one of the major spreading ways of a flu or an infectious disease, and there are many kinds of bacterial and virus may crossly infected between humans via the droplet, the droplet could be remained in any part of a human or an object, so that doctors recommend people to wash hands frequently to avoid residuals of the droplet from entering a human body through his/her nose, eyes and mouth; if an infection actually causes, the temperature of the human body would raise to form a protection mechanism, and one of the objective conditions for determining whether a fever reaction is to measure the body temperature.

At present, tools for measuring temperatures are: an underarm thermometer, a mouth thermometer, an anal thermometer, an ear thermometer, a forehead thermometer and a thermography imaging equipment, wherein the underarm thermometer, the mouth thermometer and the anal thermometer are to perform a temperature measuring processed by utilizing a property of a liquid being thermally expanded and contracted, and the ear thermometer, the forehead thermometer and the thermography imaging equipment are to utilize infrared to measure the body temperature which is well known by people; if the epidemiology of infectious disease is adopted as an issue to be concerned, the above-mentioned products, besides of the forehead thermometer and the thermography imaging equipment, would have a direct contact with the human body, which are not suitable to be applied as a means of collective testing.

However, the forehead thermometer is mainly used for measuring the body temperature within a close distance and a one by one means, so that the consuming time is relatively caused, and an additional risk of increasing the possibility of getting infected will have to take due to an overly close distance between the temperature measuring personnel and the person to be tested; the thermography imaging equipment has an advantage of rapidly measuring and determining, but the cost thereof is high and could not be prevailed on various occasions; as such, a designing concept of the present invention is to consider: whether a product capable of rapidly determining, capable of being prevailed and capable of overcoming a measuring means of being in direct contact between people, thus the present invention provides a product combining a temperature measuring tool and a wearable headset, and the product can be connected to a smart phone with a wired or a wireless means for processing relevant applications, thereby achieving an objective of rapidly testing and measuring at any desired time.

### 2. Description of Related Art

Please refer to an upper part of FIG. 1, a mobile phone capable of measuring body temperature was disclosed in 2004, the structure includes a casing, an antenna, a body temperature sensor, a control module and a display panel, wherein the antenna is disposed on a lateral side of the casing, the body temperature sensor is disposed at a distal end of the antenna and used for being in contact with a human body so as to output a temperature measuring signal, the control module disposed in the casing is used for processing the temperature measuring signal, and the display panel is used for displaying a value; based on the disclosed concept, the mobile phone capable of measuring body temperature was novel at that time, but with the ever-changing technology, the transmitting technology, the structure of a mobile phone, and the application levels of software and hardware are all well developed and could not be compared with the old days, the first generation of iphone was launched by Steve Jobs in 2007, which was three years later; accordingly, the smart phones developed in recent years, especially in 2020, can be more widely applied in terms of applications.

Please refer to a lower part of FIG. 1, an intelligent ear temperature thermometer, named as “Tympani” is launched by the Caring Things based in Massachusetts of Untied States, the device is inserted in a headset insertion hole of a smart phone, and infrared is utilized for sensing and reading the temperature of a tympanic membrane after the insertion, the data is transmitted to the smart phone via the headset insertion hole for determining; the designing concept of the device is very novel and a feature thereof is that an additional battery is not required; as such, the device has to be inserted in the headset insertion hole of the smart phone while being desired to be used, and the device has to be disposed close to the ear during every measurement; however, a problem of whether the device being properly inserted in the ear canal with a correct angle shall be concerned if the measurement was taken without any other people being there for assistance.

### SUMMARY OF THE INVENTION

According to what has been disclosed above, besides the conventional thermometer available in the markets and applied for measuring the body temperature, a temperature sensor module can also be used for measuring, wherein the temperature sensor can utilize a temperature sensing chip or infrared, moreover, the infrared is commonly adopted in the ear thermometer, the foundation of the measured temperature is relative to the hypothalamus of a brain, this is because the hypothalamus is defined as the central point of the body temperature, which means that the body temperature of the hypothalamus would firstly response to a fever so as to raise the temperature, and blood flow at the eardrum and the ambient tissues are communicated with the blood flow at a periphery close to the hypothalamus; as such, the ear thermometer is to detect the infrared emitted by the hypothalamus, and the Wein's displacement law is adopted for converting the wavelength into the temperature which is well known by people.

In view of the above, the concept of the present invention is to base on an ear temperature measuring foundation, and is aimed to overcome a direct contact in a close distance between people and to provide a rapidly determining means, so that a combination of a temperature sensor module and a headset is provided by the present invention, because the headset has properties of light in weight and capable of being precisely inserted in the ear canal by the user, a concern of whether the angel being correct while being inserted in the ear canal can be relieved, according to the present invention, the headset can be a wired or a wireless product, and the headset is able to perform a transmission to a smart phone via a headset hole, a charging hole or a wireless technology, then an virtual button of an APP software or a button module (real buttons on the headset) of the headset can be used for switching between functions of listening to music, talking or temperature measuring, lastly the relevant data can be displayed on a screen of the smart phone, or the temperature data can be uploaded to a cloud at any desired time for allowing the quarantine inspector to perform an initial determination.

Moreover, it is not a surprise that everyone has a smart phone, so that the hardware structure and the transmitting function are both well known by the users, and there are many different accessories, for example, a headset; the headset can be categorized in a hanging type, an in-ear type, a bone-conduction type, a wired type or a wireless type, which are known and used by the consumers; so that the structure of headset and the transmitting function are not further provided by the present invention for providing a clear disclosure of the technical feature of the present invention.

### BRIEF DESCRIPTION OF THE DRAWINGS

FIG. 1 is a schematic view wherein an upper part thereof showing a mobile phone having temperature measuring function and an lower part thereof showing an intelligent ear thermometer; and

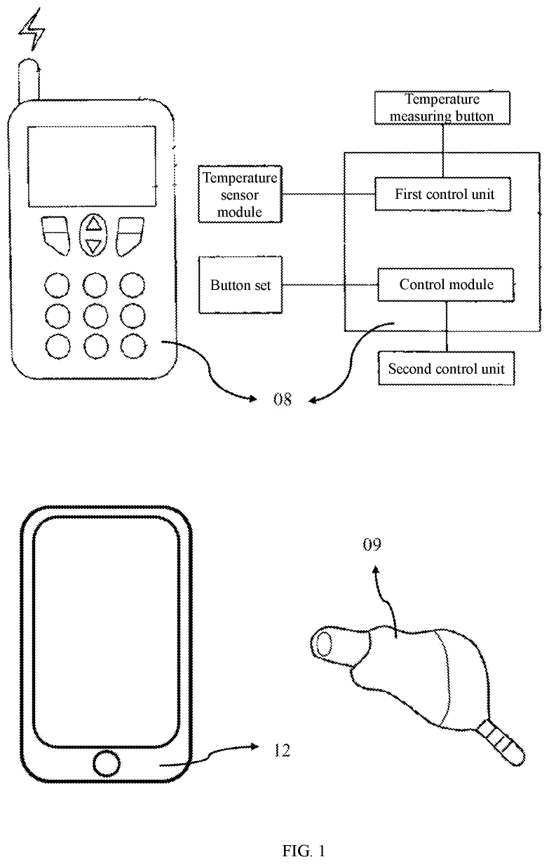

FIG. 2 is a schematic view showing the ear temperature measurement of a wearable headset according to the present invention.

### BRIEF DESCRIPTION OF CODES

10: Temperature sensor module
11: Headset
12: Smart phone
13: Headset soft cap
14: Headset insertion hole
15: Charging hole
16: Virtual button of APP software
17: Button module of headset

### DETAILED DESCRIPTION OF THE PREFERRED EMBODIMENT

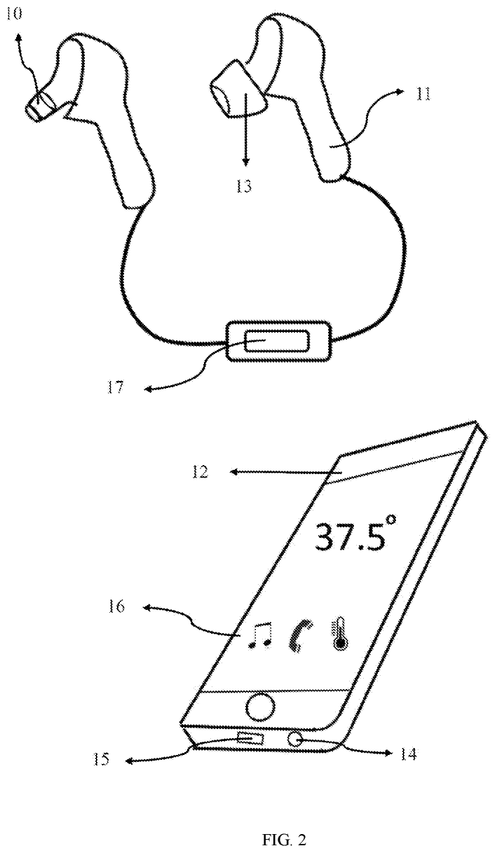

Please refer to FIG. 2, the present invention provides an ear temperature measurement of a wearable headset. According to the present invention, a first step of designing the ear temperature measurement of the wearable headset is that: a temperature sensor module 10 is disposed in a headset 11, and the module can be a temperature sensing chip or an infrared transmitting module; moreover, if a requirement of providing comfortableness is desired, a soft cap can be provided at a location where a distal end of the headset being in contact with the ear canal, and hereinafter defined as a headset soft cap 13 which is used for increasing the wearing comfortableness, the headset 11 can be a wired or wireless hanging type, plug-type, in-ear type, around-neck type or bone-conduction type, and the temperature sensor module 10 can be categorized in three types: (1) a temperature sensing chip module; (2) an infrared transmitting module; and (3) a combination of the two.

A second step is that: establishing a transmitting means, an objective is to enabling the data measured by the temperature sensor module 10 to be transmitted to a smart phone 12, and the transmitting means can be a wired or wireless means; if the wired means is adopted as the transmitting means, a headset insertion hole 14 or a charging hole 15 of the smart phone 12 can be used for transmitting data; if the wireless mean is adopted of the transmitting means, wireless means, for example NFC, Zig-Bee, Bluetooth, Wi-Fi, RFID and IrDA, can be adopted for transmitting data.

A third step is that: switching functions via a button set, wherein the button set can be a virtual button 16 of an APP software stored in the smart phone 12, or an actual button module 17 installed on a wired or a wireless headset structure so as to switch between functions of listening music, talking or temperature measuring; when the headset 11 is worn and the function is switched to the selection of temperature measuring, the temperature sensor module 10 inside the headset 11 is actuated for measuring a body temperature, and a measured result is displayed on a screen of the smart phone 12; if a situation of having a fever is determined, functions of generating warning sounds, texts or vibrations can be provided, then the measured temperature is uploaded to a cloud for allowing a quarantine inspector to perform an initial termination and track.

What is claimed is:

1. An ear temperature measurement of wearable headset, including: a headset, a temperature sensor module and a smart phone, wherein a headset soft cap being disposed at a distal end of the headset, and characterized in that: the temperature sensor module is disposed in the headset, data of the temperature sensor module is transmitted to the smart phone and displayed on a screen of the smart phone, and button sets of the headset or the smart phone are used for switching functions.

2. The ear temperature measurement of wearable headset as claimed in claim 1, wherein the headset is a wired or wireless hanging type, plug-type, in-ear type, around-neck type or bone-conduction type headset.

3. The ear temperature measurement of wearable headset as claimed in claim 1, wherein the temperature sensor module is selected from a group consisted of a temperature sensing chip module and an infrared transmitting module, or a combination of the above.

4. The ear temperature measurement of wearable headset as claimed in claim 1, wherein a transmitting means of the temperature sensor module transmitting the data to the smart phone is a wired or wireless means; if the wired means is adopted as the transmitting means, a headset insertion hole or a charging hole of the smart phone is used for transmitting data; and if the wireless mean is adopted as the transmitting means, wireless means of NFC, Zig-Bee, Bluetooth, Wi-Fi, RFID or IrDA is adopted for transmitting data.

5. The ear temperature measurement of wearable headset as claimed in claim 1, wherein, the button set is selected from a group consisted of a button module of the headset and a virtual button of an APP software stored in the smart phone.
